(19) United States
(12) Patent Application Publication
(10) Pub. No.: US 2025/0205488 A1
(43) Pub. Date: Jun. 26, 2025

(54) LOW FREQUENCY THERAPEUTIC APPARATUS, CONTROL METHOD, AND CONTROL RECORDING MEDIUM

(71) Applicant: OMRON HEALTHCARE Co., Ltd., Kyoto (JP)

(72) Inventor: Tetsuya SATO, Kyoto (JP)

(21) Appl. No.: 19/075,553

(22) Filed: Mar. 10, 2025

Related U.S. Application Data
(63) Continuation of application No. PCT/JP2023/040266, filed on Nov. 8, 2023.

(30) Foreign Application Priority Data
Mar. 8, 2023 (JP) 2023-035943

(51) Int. Cl. A61N 1/36; A61N 1/04

(52) U.S. Cl. CPC A61N 1/36034; A61N 1/0492; A61N 1/0452

(57) ABSTRACT

A low-frequency treatment device according to one aspect of the present invention includes a pad unit, an operation unit that receives an operation from a user, and a processor. The processor causes the pad unit to supply a low-frequency pulse current that provides electrical stimuli for treatment. The processor causes the pad unit to supply a notification pulse current that provides electrical stimuli, different from the electrical stimuli for treatment, to notify the user of receipt of the operation to the operation unit.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040266, filed Nov. 8, 2023, which application claims priority to Japanese Patent Application No. 2023-035943, filed Mar. 8, 2023, which applications are incorporated herein by reference in their entireties.

### TECHNICAL FIELD

The present invention relates to a low-frequency treatment device, a control method, and a control recording medium.

### BACKGROUND ART

Conventionally, a low-frequency treatment device is known that performs treatment of, for example, relieving shoulder stiffness of a user by attaching a pad including a conductive layer to the body of the user and supplying a low-frequency pulse current to the body.

Patent Document 1 describes a frequency treatment device that notifies a patient through a speaker when treatment has ended or the like. Patent Document 2 describes an electronic treatment device that urges a user to increase output by sound or display each time a predetermined time has elapsed since a start of output of modulated waves.

Patent Document 3 describes a low-frequency treatment device that causes a pad unit to supply a low-frequency pulse current that provides electrical stimuli for treatment and notifies a user of progress of the treatment. Patent Document 4 describes an electrical stimulation device that provides notification signals regarding operation states such as a charge level of the electrical stimulation device, a status of a timer module, and a warning state.

### CITATION LIST

### Patent Literature

Patent Document 1: JP 2019-170873 A
Patent Document 2: JP 2011-015723 A
Patent Document 3: JP 2022-018442 A
Patent Document 4: JP 2015-536748 T

### SUMMARY OF INVENTION

### Technical Problem

In devices such as low-frequency treatment devices, feedback that notifies a user that an operation performed by the user has been received is important for improving usability of the device. Here, an integrated configuration is assumed in which an operation unit for operating a body of a low-frequency treatment device is provided on the body of the low-frequency treatment device, which is attached to a human body when in use. In such a configuration, for example, even when a display is provided on the body of the low-frequency treatment device and feedback on the operation is provided through a screen display, it may be difficult for the user to visually recognize the display. In particular, in a low-frequency treatment device that is attached to the back or the like of the user, it is difficult for the user to visually recognize the display attached to the back or the like.

It is also conceivable to provide audio feedback on the operation through a speaker. However, when using the low-frequency treatment device for treatment away from home, such as at a workplace, the user feels resistance to sound notification (the user may be worried about the eyes of others). In particular, in a low-frequency treatment device that is attached to a user's back or the like, the low-frequency treatment device is away from user's ears and is often covered with clothing or the like.

Therefore, the sound output needs to be loud enough for the user to hear, which may cause the user to feel strong resistance. In addition, installing the speaker in the body of the low-frequency treatment device leads to an increase in size and cost of the device.

It is also conceivable to provide feedback on the operation by vibration using a vibrator. In the low-frequency treatment device, it is important to keep electrodes in contact with appropriate positions on the user's body, but vibration can cause the electrodes to shift from their appropriate positions or come off. In particular, in the low-frequency treatment device that is attached to the user's back or the like, the user's back or the like is an area where the user hardly feels vibration, so intensity of the vibration needs to be increased so that the user can feel the vibration, which can make the electrodes more likely to shift from their positions or come off. In addition, installing the vibrator in the body of the low-frequency treatment device leads to an increase in size and cost of the device.

It is also conceivable to wirelessly connect the low-frequency treatment device to an information terminal such as a smartphone and provide feedback on the operation via a screen display of the information terminal. However, when wireless communication is used, delays in notification due to communication delays and interference from communication radio waves that can affect low-frequency treatment can be a problem. In addition, installing the wireless communication module in the body of the low-frequency treatment device leads to an increase in size and cost of the device.

Thus, in particular, with the integrated low-frequency treatment device that is attached to an area that is difficult for the user to visually recognize, it is difficult to operate it without feeling resistance when the user is away from home or to provide feedback on the operation while preventing misalignment or the like, which makes it difficult to improve usability. Furthermore, means for solving such problems are not disclosed in Patent Documents 1 to 4.

The present invention has been made in view of such circumstances in one aspect, and an object of the present invention is to provide a technology that can improve usability.

### Solution to Problem

The present invention employs the following configurations to solve the above-described problems.

### 1

A low-frequency treatment device including a pad unit configured to supply a pulse current to an object to be in contact, an operation unit configured to receive an operation from a user, and a control unit configured to cause the pad unit to supply a low-frequency pulse current that provides an electrical stimulus for treatment and a notification pulse current that provides another electrical stimulus configured to notify the user of receipt of the operation, the other electrical stimulus being different from the electrical stimulus for treatment.

According to (1), in addition to the low-frequency pulse current that provides the electrical stimulus for treatment, the notification pulse current that provides the other electrical stimulus to notify the user of the receipt of the operation from the user is supplied to the user. Therefore, even when the user has difficulty visually recognizing the low-frequency treatment device, the user can perceive and distinguish the notification pulse current from the low-frequency pulse current for treatment and can clearly recognize that the operation he or she performed has been received by the low-frequency treatment device. This improves usability of the low-frequency treatment device.

### 2

The low-frequency treatment device according to (1), in which the operation unit is integrally formed with the pad unit.

According to (2), in the low-frequency treatment device in which the operation unit is integrally formed with the pad unit and it may be difficult to visually recognize the operation unit, the user can clearly recognize that the operation he or she performed has been received by the low-frequency treatment device.

### 3

The low-frequency treatment device according to (2), in which the low-frequency treatment device is attached to the back of a human body.

According to (3), in the low-frequency treatment device in which the operation unit is located on the back of the human body and it is difficult to visually recognize the operation unit, the user can clearly recognize that the operation he or she performed has been received by the low-frequency treatment device.

### 4

The low-frequency treatment device according to any one of (1) to (3) in which the control unit causes the pad unit to immediately supply the notification pulse current in response to the receipt of the operation.

According to (4), the user can intuitively recognize that the notification pulse current indicates the receipt of the operation performed by the user.

### 5

The low-frequency treatment device according to any one of (1) to (4), in which the control unit causes the pad unit to supply the notification pulse current that changes depending on a type of the operation received.

According to (5), the user can easily recognize whether the operation he or she intended has been received.

### 6

The low-frequency treatment device according to (5), in which the control unit, when receiving an operation instructing an increase of stimulus intensity of the low-frequency pulse current, causes the pad unit to supply the notification pulse current having a stimulus intensity that increases over time, and when receiving an operation instructing a decrease of stimulus intensity of the low-frequency pulse current, causes the pad unit to supply the notification pulse current having a stimulus intensity that decreases over time.

According to (6), the user can intuitively distinguish a case in which the operation instructing the increase of the stimulus intensity of the low-frequency pulse current has been received from a case in which the operation instructing the decrease of the stimulus intensity of the low-frequency pulse current has been received.

### 7

The low-frequency treatment device according to any one of (1) to (6), in which the operation unit is configured to receive a switching operation configured to switch at least one of a stimulus intensity and a type of the low-frequency pulse current to be supplied, and the control unit switches at least one of the stimulus intensity and the type of the low-frequency pulse current in response to the switching operation received, and causes the pad unit to supply the notification pulse current that changes in response to at least one of the stimulus intensity and the type of the low-frequency pulse current after switching.

According to (7), the user can easily recognize the stimulus intensity and the type of the low-frequency pulse current after switching by his/her own switching operation.

### 8

A method for controlling a low-frequency treatment device including a pad unit configured to supply a pulse current to an object to be in contact, an operation unit configured to receive an operation from a user, and a processor, the method including causing the pad unit to supply a low-frequency pulse current that provides an electrical stimulus for treatment and a notification pulse current that provides another electrical stimulus configured to notify the user of receipt of the operation, the other electrical stimulus being different from the electrical stimulus for treatment.

According to (8), as in (1), the usability of the low-frequency treatment device can be improved.

### 9

A non-transitory computer-readable storage medium storing a control recording medium for a low-frequency treatment device including a pad unit configured to supply a pulse current to an object to be in contact, an operation unit configured to receive an operation from a user, and a processor, the control recording medium causing the processor to execute an operation including causing the pad unit to supply a low-frequency pulse current that provides an electrical stimulus for treatment and a notification pulse current that provides another electrical stimulus configured to notify the user of receipt of the operation, the other electrical stimulus being different from the electrical stimulus for treatment.

According to (9), as in (1), the usability of the low-frequency treatment device can be improved.

### Advantageous Effects of Invention

According to the present invention, a technology can be provided that can improve usability.

### BRIEF DESCRIPTION OF DRAWINGS

FIG. 1 is a plan view illustrating a schematic configuration of a low-frequency treatment device 100.

FIG. 2 is a schematic diagram illustrating an example of a state in which the low-frequency treatment device 100 is attached to a body 50 of a user.

FIG. 3 is a block diagram illustrating an example of a hardware configuration of a main body unit 20.

FIG. 4 is a diagram illustrating an example of a pulse voltage applied by the main body unit 20 to a pad unit 10.

FIG. 5 is a diagram illustrating an example of a low-frequency pulse current and a notification pulse current supplied by the low-frequency treatment device 100.

FIG. 6 is a diagram illustrating examples of notification pulse currents according to types of operations.

### DESCRIPTION OF EMBODIMENTS

Embodiments according to one aspect of the present invention will be described below based on the drawings.

### Application Example

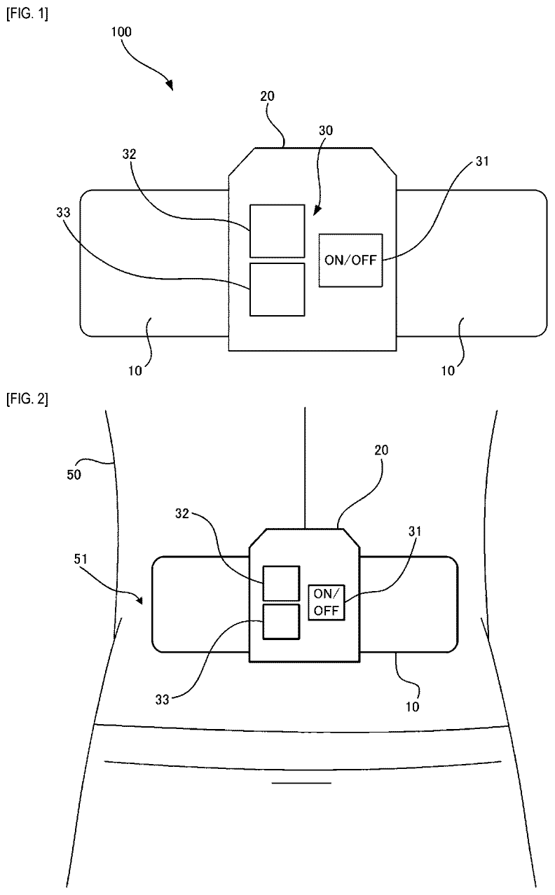

FIG. 1 is a plan view illustrating a schematic configuration of a low-frequency treatment device 100.

The low-frequency treatment device 100 is a cordless type low-frequency treatment device for performing treatment or the like by supplying a low-frequency pulse current to provide electrical stimuli to a user. A frequency of the low-frequency pulse current is not limited to a specific frequency, but is, for example, about 1 to 1200 Hz. The low-frequency treatment device 100 includes a pad unit 10 that is an attachment unit that is attached to a human body (a user's body) and receives power, and a main body unit 20 that is connected to the pad unit 10 and controls the power supplied to the pad unit 10.

The pad unit 10 can be attached to the user's body. The pad unit 10 includes a conductive layer (not illustrated) that supplies a low-frequency pulse current to the user, and can supply a pulse current to the user's body, which is an attachment portion (object to be in contact), using power supplied from the main body unit 20. The pad unit 10 is formed, for example, by layering carbon layers, which are conductors, by printing on a surface of a base material made of a soft synthetic resin, and these carbon layers serve as the conductive layer.

A holder (not illustrated) is integrally provided with the pad unit 10, and the main body unit 20 is fixed to the holder, thereby connecting the pad unit 10 and the main body unit 20. Since the pad unit 10 is preferably replaceable (consumable), the main body unit 20 is preferably attachable to and detachable from the holder.

The main body unit 20 includes, for example, a box-shaped housing, and an operation unit 30 is located on a surface of the housing (a surface facing a side opposite to a human body side when the main body unit 20 is attached to the human body). The operation unit 30 includes, for example, input devices that receive operation input from the user. The input devices can be achieved, for example, by keys and the like.

In the example in FIG. 1, as the input devices included in the operation unit 30, illustrated are a power key 31 for turning a power source of the low-frequency treatment device 100 on and off, and a first key 32 and a second key 33 for adjusting an intensity of stimuli applied to the body by the low-frequency treatment device 100. Hereinafter, the first key 32 and the second key 33 are also collectively referred to as stimulus adjustment keys.

As described with reference to FIG. 1, the operation unit 30 is integrally formed with the pad unit 10. The operation unit 30 being integrally formed with the pad unit 10 means, for example, that the operation unit 30 and the pad unit 10 are not separate units connected by a cable or the like, but are fixed to each other as a single device.

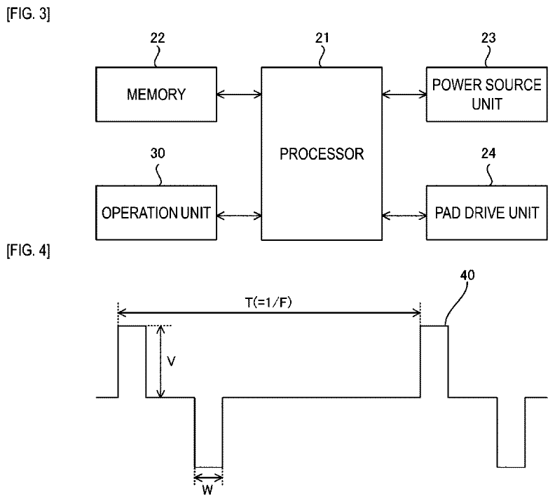

FIG. 2 is a schematic diagram illustrating an example of a state in which the low-frequency treatment device 100 is attached to a body 50 of a user. As illustrated in FIG. 2, the pad unit 10 is attached to, for example, a lumbar region 51 of the body 50 of the user. The lumbar region 51 is an example of the back of a human body. The pad unit 10 is in a shape of a band extending in a right-left direction of the user so as to cover a wide area of the lumbar region 51. FIG. 2 illustrates a recommended attachment state in which the low-frequency treatment device 100 is attached to the lumbar region 51 for use.

Since the low-frequency treatment device 100 is attached to the back of the body 50 (lumbar region 51), the user can operate the operation unit 30, for example, with fingers of a hand, but it is difficult for the user to visually recognize the operation unit 30.

### Hardware Configuration of Main Body Unit 20

FIG. 3 is a block diagram illustrating an example of a hardware configuration of the main body unit 20. The main body unit 20 includes a processor 21, a memory 22, a power source unit 23, a pad drive unit 24, and the operation unit 30.

The processor 21 is, for example, a processor such as a central processing unit (CPU) or a micro processing unit (MPU). The processor 21 controls operations of components of the low-frequency treatment device 100 by reading out and executing a recording medium stored in the memory 22. Note that the processor 21 may be a combination of multiple processors. The processor 21 is an example of a control unit according to the present invention.

The memory 22 is achieved by a random access memory (RAM), a read only memory (ROM), a flash memory, or the like. The memory 22 stores recording mediums executed by the processor 21, data used by the processor 21, and the like.

The power source unit 23 supplies power to components of the low-frequency treatment device 100. A power source may be, for example, a dry cell or a rechargeable battery such as a lithium ion battery or a nickel hydrogen battery, which generates a drive voltage to provide stabilized battery voltage to the components. The power source unit 23 is not limited to a battery, and may supply power to the components of the low-frequency treatment device 100 using power supplied from a home power source or the like. In this case, an external power source such as the home power source may be connected to the main body unit 20 by a cable or the like.

The pad drive unit 24 applies a pulse voltage to the pad unit 10, thereby controlling supply of a pulse current to the user by the pad unit 10. The pad drive unit 24 is controlled by the processor 21.

The processor 21 causes the pad unit 10 to supply a low-frequency pulse current that provides electrical stimuli for treatment. When the user operates the operation unit 30, the processor 21 causes the pad unit 10 to supply a notification pulse current that provides electrical stimuli for notifying the user of receipt of the operation by stimulating the skin or muscles different from the electrical stimuli for treatment by the low-frequency pulse current.

The notification pulse current is a pulse current that provides electrical stimuli different from the electrical stimuli for treatment. To be specific, the notification pulse current is a pulse current different from the low-frequency pulse current in at least one of a pulse width, an amplitude, and a period.

For example, the notification pulse current is a pulse current having a pulse width narrower than a pulse width of the low-frequency pulse current. This makes it easier to perceive and distinguish the notification pulse current from the low-frequency pulse current for treatment having a relatively wide pulse width. However, the notification pulse current may be a pulse current having a pulse width wider than the pulse width of the low-frequency pulse current. Also in this case, the user can perceive and distinguish the notification pulse current from the low-frequency pulse current for treatment.

The notification pulse current may be a pulse current having an amplitude smaller than an amplitude of the low-frequency pulse current. This makes it easier to perceive and distinguish the notification pulse current from the low-frequency pulse current while suppressing an excessive burden on the user. However, the notification pulse current may be a pulse current having an amplitude larger than the amplitude of the low-frequency pulse current. Also in this case, the user can perceive and distinguish the notification pulse current from the low-frequency pulse current for treatment.

The notification pulse current may be a pulse current having a period shorter than a period of the low-frequency pulse current (i.e., a higher frequency pulse current). This makes it easier to perceive and distinguish the notification pulse current from the low-frequency pulse current for treatment having a relatively long period. However, the notification pulse current may be a pulse current having a period shorter than the period of the low-frequency pulse current. Also in this case, the user can perceive and distinguish the notification pulse current from the low-frequency pulse current for treatment.

Thus, the low-frequency treatment device 100 supplies the user with the notification pulse current that provides the electrical stimuli to notify the user of the receipt of the operation from the user, in addition to the low-frequency pulse current that provides the electrical stimuli for treatment. This allows, even when it is difficult for the user to visually recognize the main body unit 20 of the low-frequency treatment device 100, the user to perceive and distinguish the notification pulse current from the low-frequency pulse current for treatment, and clearly recognize that the operation he or she performed has been received by the low-frequency treatment device 100.

In addition, unlike when feedback on the operation is provided by sound through a speaker, even when performing treatment with the low-frequency treatment device 100 away from home, such as at a workplace, the user can operate the low-frequency treatment device 100 without feeling any resistance (without worrying about the eyes of others). In addition, since the low-frequency treatment device 100 does not need to be provided with a speaker, the device can be made smaller and less expensive.

In addition, unlike when feedback on the operation is provided by vibration from a vibrator, direct vibration associated with the feedback on the operation is not generated, so that misalignment or detachment of the pad unit 10 can be suppressed. In addition, since the low-frequency treatment device 100 does not need to be provided with a vibrator, the device can be made smaller and less expensive.

In addition, unlike when feedback on the operation is provided by a screen display of an information terminal wirelessly connected to the low-frequency treatment device 100, delays in notification due to communication delays and interference from communication radio waves that can affect low-frequency treatment can be prevented. In addition, since the low-frequency treatment device 100 does not need to be provided with a wireless communication module, the device can be made smaller and less expensive.

### Pulse Voltage Applied by Main Body Unit 20 to Pad Unit 10

FIG. 4 is a diagram illustrating an example of a pulse voltage applied by the main body unit 20 to the pad unit 10. A pulse voltage waveform 40 illustrated in FIG. 4 is an example of a waveform of the pulse voltage applied by the pad drive unit 24 of the main body unit 20 to the pad unit 10.

Parameters of the pulse voltage waveform 40 include an amplitude (voltage) V, a pulse width W, and a pulse period T (pulse frequency F=1/T). The main body unit 20 can change treatment contents for the user by changing at least one of the parameters.

To be specific, the main body unit 20 boosts a power supply voltage to a predetermined voltage and adjusts the boosted voltage to a voltage corresponding to a set amplitude. For example, the main body unit 20 can adjust the amplitude V of the pulse voltage at a predetermined number of levels (10-stage levels) according to an instruction from the user. When the main body unit 20 receives a setting input with a certain level from the user, the main body unit 20 generates, based on the amplitude V corresponding to that level, a treatment waveform (pulse waveform) corresponding to a treatment mode and outputs the treatment waveform to the pad unit 10.

The low-frequency treatment device 100 is provided with multiple treatment modes in advance. The treatment modes include, for example, “massage”, “tap”, “press”, and “sweep”. The main body unit 20 changes a waveform of a pulse voltage applied to the pad unit 10 to provide electrical stimulation corresponding to various modes through the pad unit 10.

In addition to the low-frequency pulse current that provides the electrical stimuli for treatment, the main body unit 20 causes the pad unit 10 to supply a notification pulse current that provides electrical stimuli to notify the user of the receipt of an operation. For example, the main body unit 20 applies a pulse voltage for generating a low-frequency pulse current and a pulse voltage for generating a notification pulse current to the pad unit 10. Each of these pulse voltages is generated based on a predetermined waveform.

### Low-Frequency Pulse Current and Notification Pulse Current Supplied by Low-Frequency Treatment Device 100

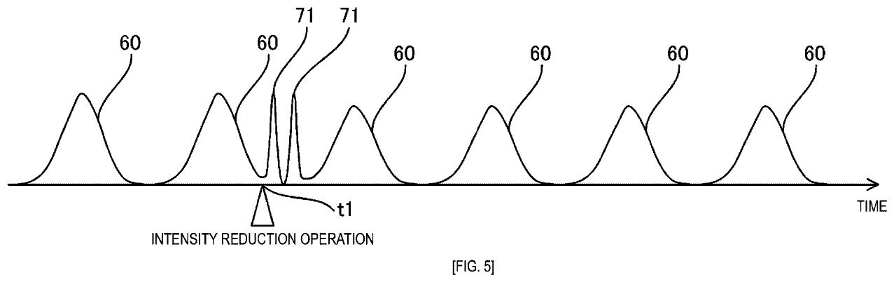

FIG. 5 is a diagram illustrating an example of the low-frequency pulse current and the notification pulse current supplied by the low-frequency treatment device 100. In FIG. 5, a horizontal axis represents time and a vertical axis represents an amplitude (voltage) of the pulse current.

The low-frequency treatment device 100 causes the pad unit 10 to supply, for example, a low-frequency pulse current 60 illustrated in FIG. 5 to the user. The low-frequency pulse current 60 is a pulse current that provides electrical stimuli for treatment. The low-frequency pulse current 60 is a pulse current that is repeated with the same amplitude, pulse width, and period unless a switching operation is performed by the user.

It is assumed that at timing t1, an intensity reduction operation (e.g., pressing the second key 33) is performed on the operation unit 30 to reduce the stimulus intensity of the low-frequency pulse current 60 by one step. In this case, the low-frequency treatment device 100 reduces the stimulus intensity of the low-frequency pulse current 60 after timing t1 (in this example, reduces the amplitude), and causes the pad unit 10 to immediately supply a notification pulse current 71 to the user at timing t1. To cause the pad unit 10 to immediately supply a pulse current at a certain point in time means, for example, to cause the pad unit 10 to supply a pulse current from that point in time without performing a process of waiting for time to elapse. Alternatively, to cause the pad unit 10 to immediately supply a pulse current at a certain point in time means to perform a process of waiting for a short period of time (e.g., 100 ms or less) that is not substantially perceived by the user to elapse from the certain point in time, and then cause the pad unit 10 to immediately supply a pulse current.

The stimulus intensity of the low-frequency pulse current 60 is, for example, the intensity of stimulus to the user that changes depending on at least one of the amplitude, the pulse width, and the period of the low-frequency pulse current 60. The greater the amplitude of the low-frequency pulse current 60, the higher the stimulus intensity of the low-frequency pulse current 60. The longer the pulse width of the low-frequency pulse current 60, the higher the stimulus intensity of the low-frequency pulse current 60. The shorter the period of the low-frequency pulse current 60, the higher the stimulus intensity of the low-frequency pulse current 60. Here, a case in which the stimulus intensity of the low-frequency pulse current 60 is adjusted by changing the amplitude of the low-frequency pulse current 60 will be described.

The notification pulse current 71 is a pulse current that provides electrical stimuli to notify the user of receipt of an operation, which is different from electrical stimuli for treatment by the low-frequency pulse current 60. In the example in FIG. 5, the notification pulse current 71 is a pulse current that has a smaller amplitude, a narrower pulse width, and a shorter period than the low-frequency pulse current 60. The notification pulse current 71 is a pulse current that is repeated twice in a short period of time (e.g., a period shorter than the period of the low-frequency pulse current 60).

As an example, the main body unit 20 can supply the notification pulse current 71 from the pad unit 10 by turning on the application of voltage to the pad unit 10 for 0.5 seconds, then turning off the application of voltage for 0.5 seconds, and then turning on the application of voltage for 0.5 seconds.

Thus, by supplying, in addition to the low-frequency pulse current 60, the notification pulse current 71 that provides the electrical stimuli different from the low-frequency pulse current 60, the user can perceive the notification pulse current 71 as electrical stimuli different from the low-frequency pulse current 60.

### Notification Pulse Current According to Type of Operation

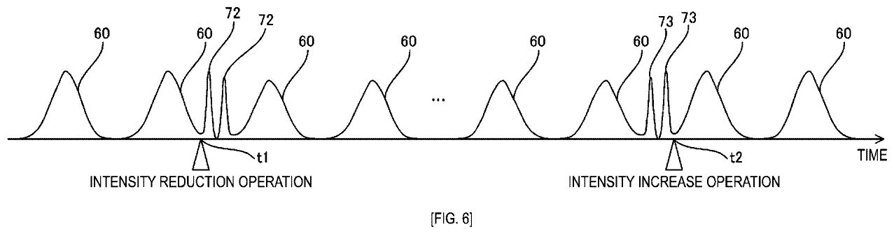

FIG. 6 is a diagram illustrating examples of notification pulse currents according to types of operations. The processor 21 may control the pad unit 10 to supply different notification pulse currents depending on the types of operations received from the user by the operation unit 30.

In the example illustrated in FIG. 6, similar to the example in FIG. 5, it is assumed that at timing t1, an intensity reduction operation is performed on the operation unit 30 to reduce the stimulus intensity of the low-frequency pulse current 60 by one step. In addition, in the example illustrated in FIG. 6, it is assumed that at timing t2, an intensity increase operation is performed on the operation unit 30 to increase the stimulus intensity of the low-frequency pulse current 60 by one step.

The processor 21 reduces the stimulus intensity of the low-frequency pulse current 60 (in this example, reduces the amplitude) from timing t1 onwards when the intensity reduction operation is performed, and causes the pad unit 10 to immediately supply a notification pulse current 72 to the user at timing t1. The notification pulse current 72 is a pulse current that repeats twice in a short period of time, similar to the notification pulse current 71 illustrated in FIG. 5, but a second pulse current has a smaller amplitude than a first pulse current.

Further, the processor 21 increases the stimulus intensity of the low-frequency pulse current 60 (in this example, increases the amplitude) from timing t2 onwards when the intensity increase operation is performed, and causes the pad unit 10 to immediately supply a notification pulse current 73 to the user at timing t2. The notification pulse current 73 is a pulse current that is repeated twice in a short period of time, similar to the notification pulse current 71 illustrated in FIG. 5, but a second pulse current has a larger amplitude than a first pulse current.

As described with reference to FIG. 6, when the processor 21 receives an intensity reduction operation to reduce the stimulus intensity (reduce the amplitude) of the low-frequency pulse current 60, the processor 21 supplies the notification pulse current 72 in which the stimulus intensity decreases over time (monotonically decreases). This allows the user to intuitively recognize that the intensity reduction operation has been received. Further, when the user mistakenly performs the intensity reduction operation while intending to perform another operation such as the intensity increase operation, the user can intuitively recognize that he or she has mistakenly performed the intensity reduction operation.

When the processor 21 receives the intensity increase operation for increasing the stimulus intensity of the low-frequency pulse current 60, the processor 21 supplies the notification pulse current 73 that increases the stimulus intensity. This allows the user to intuitively recognize that the intensity increase operation has been received. Further, when the user mistakenly performs the intensity increase operation while intending to perform another operation such as the intensity reduction operation, the user can intuitively recognize that he or she has mistakenly performed the intensity increase operation.

### Notification Pulse Currents 72 and 73 when Stimulus Intensity of Low-Frequency Pulse Current 60 is Adjusted by Changing Pulse Width

Although the case in which the stimulus intensity of the low-frequency pulse current 60 is adjusted by changing the amplitude of the low-frequency pulse current 60 has been described, the stimulus intensity of the low-frequency pulse current 60 may be adjusted by changing a length of the pulse width of the low-frequency pulse current 60. In this case, the intensity increase operation (e.g., pressing the first key 32) is an operation to increase the pulse width of the low-frequency pulse current 60, and the intensity reduction operation (e.g., pressing the second key 33) is an operation to decrease the pulse width of the low-frequency pulse current 60.

In this case, the notification pulse current 72 corresponding to the intensity increase operation may be, for example, a pulse current that is repeated twice in a short period of time, similar to the notification pulse current 71, and the second pulse may have a longer pulse width than the first pulse. The notification pulse current 72 corresponding to the intensity reduction operation may be, for example, a pulse current that is repeated twice in a short period of time, similar to the notification pulse current 71, and the second pulse may have a shorter pulse width than the first pulse.

### Notification Pulse Currents 72 and 73 when Stimulus Intensity of Low-Frequency Pulse Current 60 is Adjusted by Changing Period

The stimulus intensity of the low-frequency pulse current 60 may be adjusted by changing the period of the low-frequency pulse current 60. In this case, the intensity increase operation (e.g., pressing the first key 32) is an operation to shorten the period of the low-frequency pulse current 60, and the intensity reduction operation (e.g., pressing the second key 33) is an operation to lengthen the period of the low-frequency pulse current 60.

In this case, the notification pulse current 72 corresponding to the intensity increase operation is, for example, a pulse current that is repeated three times in a short period of time, and an interval between a second pulse and a third pulse may be shorter than an interval between a first pulse and the second pulse. The notification pulse current 72 corresponding to the intensity reduction operation may be, for example, a pulse current in which an interval between the second pulse and the third pulse is longer than an interval between the first pulse and the second pulse.

### Modified Example of Method of Generating Pulse Currents

Although the configuration has been described in which the main body unit 20 applies to the pad unit 10 the pulse voltage for generating the low-frequency pulse current and the pulse voltage for generating the notification pulse current, this configuration is just an example. For example, the main body unit 20 may apply to the pad unit 10 a pulse voltage based on a waveform by combining a waveform of the low-frequency pulse current and a waveform of the notification pulse current.

### Modified Example of Notification Pulse Current

As specific examples of the notification pulse current, the pulse currents that have the pulse width shorter than the pulse width of the low-frequency pulse current, the period shorter than the period of the low-frequency pulse current, and the amplitude smaller than the amplitude of the low-frequency pulse current, respectively, and that are repeated mainly twice, have been described. However, the notification pulse current is not limited to these, and may be any type of pulse current that is distinguishable from the low-frequency pulse current.

For example, the notification pulse current may be a pulse current that is supplied only once without being repeated, or may be a pulse current that is repeated three or more times. The notification pulse current may be a pulse current that is repeated while changing at least one of an amplitude, a pulse width, and a period. For example, the notification pulse current may be a pulse current having a specific rhythm that leaves an impression on the user.

### Modified Example of Feedback on Operation

Although the configuration has been described in which different notification pulse currents are supplied according to the types of operations on the operation unit 30 (intensity increase operation and intensity reduction operation), the processor 21 may be configured to supply different notification pulse currents corresponding to the stimulus intensities of the low-frequency pulse current after switching, for example, in response to the intensity increase operation and the intensity reduction operation.

### Modified Example of Operation on Operation Unit 30

Although the intensity increase operation and the intensity reduction operation have been described as operations that trigger the supply of the notification pulse current, the operations that trigger the supply of the notification pulse current are not limited to these. For example, the processor 21 may also supply the notification pulse current when power is turned on or off using the power key 31.

The operation unit 30 may receive operations other than pressing the power key 31, the first key 32, and the second key 33. For example, the pad unit 10 may be capable of supplying multiple types of low-frequency pulse currents that provide electrical stimuli for treatment, and the operation unit 30 may be capable of receiving an operation for instructing switching of the type of the low-frequency pulse current supplied by the pad unit 10. In this case, each time the processor 21 receives an operation instruction to switch the type of the low-frequency pulse current supplied by the pad unit 10, the processor 21 switches the type of the low-frequency pulse current supplied by the pad unit 10 and causes the pad unit 10 to supply the notification pulse current. At this time, the processor 21 may cause the pad unit 10 to supply a different notification pulse current depending on the type of the low-frequency pulse current after switching.

### Configuration with Output Devices that Output Information to User, and the Like

The low-frequency treatment device 100 may include an output device that outputs information to the user, and the like. The output device can be achieved, for example, by a display, a speaker, or the like. Both the input device and the output device may be achieved by a touch panel or the like. For example, keys as the input devices may be achieved by software keys displayed on a touch panel integrated with a display.

### Feedback on Operation According to Attachment State

When the processor 21 receives an operation from the user on the operation unit 30, the processor 21 may determine whether the low-frequency treatment device 100 is attached to the user. Whether the low-frequency treatment device 100 is attached to the user can be determined by, for example, a contact sensor or the like provided on the pad unit 10.

A situation in which the user operates the operation unit 30 when the low-frequency treatment device 100 is not attached to the user is, for example, a situation in which the user turns on the power, performs preliminary settings, or the like before attaching the low-frequency treatment device 100. In such a situation, the user is likely to be able to visually recognize the operation unit 30. In addition, such a situation is likely to be a situation in which the user does not need to be concerned about the eyes of others, such as a situation immediately before the user attaches the low-frequency treatment device 100 at home.

The processor 21 provides feedback on the operation by supplying a notification pulse current when the low-frequency treatment device 100 is attached to the user, and provides feedback on the operation by an output device such as a display or a speaker when the low-frequency treatment device 100 is not attached to the user.

Thus, when the low-frequency treatment device 100 is not attached to the user, that is, in a situation where the electrical stimuli by the notification pulse current is not correctly transmitted to the user, feedback on the operation can be provided to the user, such as through a screen display or sound output, even when the low-frequency treatment device 100 is not attached to the user. In addition, it is possible to prevent the user from receiving unintended electrical stimuli due to the notification pulse current being supplied while the low-frequency treatment device 100 is attached to the user.

### Control Recording Medium

A control recording medium for the low-frequency treatment device 100 is stored in a non-transitory storage medium from which the recording medium can be read by a computer. Such a “computer-readable storage medium” includes, for example, an optical medium such as a compact disc-ROM (CD-ROM), a magnetic storage medium such as a universal serial bus (USB) memory or a memory card. Additionally, such a recording medium can also be provided by downloading via a network such as the Internet.

Although various embodiments have been described above, it is needless to say that the present invention is not limited to such examples. It will be apparent to those skilled in the art that various changes and modifications can be made within the scope of the claims, and it is understood that these naturally belonged within the technical scope of the present invention. Further, components of the above-described embodiments may be combined as desired within a range that does not depart from the spirit of the present invention.

This application is based on a Japanese patent application filed on Mar. 8, 2023 (JP 2023-035943), the contents of which are incorporated herein by reference.

### REFERENCE NUMERALS LIST

10 Pad unit
20 Main body unit
21 Processor
22 Memory
23 Power source unit
24 Pad drive unit
30 Operation unit
31 Power key
32 First key
33 Second key
40 Pulse voltage waveform
50 Body
51 Lumbar region
60 Low-frequency pulse current
71 to 73 Notification pulse current
100 Low-frequency treatment device
t1, t2 Timing

1. A low-frequency treatment device comprising:
a pad unit configured to supply a pulse current to an object to be in contact;
an operation unit configured to receive an operation from a user; and
a control unit configured to cause the pad unit to supply a low-frequency pulse current that provides an electrical stimulus for treatment and a notification pulse current that provides another electrical stimulus configured to notify the user of receipt of the operation, the other electrical stimulus being different from the electrical stimulus for treatment, wherein
the control unit,
when receiving an operation instructing an increase of stimulus intensity of the low-frequency pulse current, causes the pad unit to supply the notification pulse current having a stimulus intensity that increases over time, and
when receiving an operation instructing a decrease of stimulus intensity of the low-frequency pulse current, causes the pad unit to supply the notification pulse current having a stimulus intensity that decreases over time.

2. The low-frequency treatment device according to claim 1, wherein the operation unit is integrally formed with the pad unit.

3. The low-frequency treatment device according to claim 1, wherein the low-frequency treatment device is attached to the back of a human body.

4. The low-frequency treatment device according to claim 1, wherein the control unit causes the pad unit to immediately supply the notification pulse current in response to the receipt of the operation.

5. The low-frequency treatment device according to claim 1, wherein
the operation unit is configured to receive a switching operation configured to switch at least one of a stimulus intensity and a type of the low-frequency pulse current to be supplied, and
the control unit switches at least one of the stimulus intensity and the type of the low-frequency pulse current in response to the switching operation received, and causes the pad unit to supply the notification pulse current that changes in response to at least one of the stimulus intensity and the type of the low-frequency pulse current after switching.

6. A method for controlling a low-frequency treatment device including a pad unit configured to supply a pulse current to an object to be in contact, an operation unit configured to receive an operation from a user, and a processor, the method comprising:
causing the pad unit to supply a low-frequency pulse current that provides an electrical stimulus for treatment and a notification pulse current that provides another electrical stimulus configured to notify the user of receipt of the operation, the other electrical stimulus being different from the electrical stimulus for treatment,
when receiving an operation instructing an increase of stimulus intensity of the low-frequency pulse current, causing the pad unit to supply the notification pulse current having a stimulus intensity that increases over time, and
when receiving an operation instructing a decrease of stimulus intensity of the low-frequency pulse current, causing the pad unit to supply the notification pulse current having a stimulus intensity that decreases over time.

7. A non-transitory computer-readable storage medium storing a control recording medium for a low-frequency treatment device including a pad unit configured to supply a pulse current to an object to be in contact, an operation unit configured to receive an operation from a user, and a processor, the control recording medium causing the processor to execute an operation comprising:
causing the pad unit to supply a low-frequency pulse current that provides an electrical stimulus for treatment and a notification pulse current that provides another electrical stimulus configured to notify the user of receipt of the operation, the other electrical stimulus being different from the electrical stimulus for treatment,
when receiving an operation instructing an increase of stimulus intensity of the low-frequency pulse current, causing the pad unit to supply the notification pulse current having a stimulus intensity that increases over time, and
when receiving an operation instructing a decrease of stimulus intensity of the low-frequency pulse current, causing the pad unit to supply the notification pulse current having a stimulus intensity that decreases over time.

8. The low-frequency treatment device according to claim 2, wherein
the operation unit is configured to receive a switching operation configured to switch at least one of a stimulus intensity and a type of the low-frequency pulse current to be supplied, and
the control unit switches at least one of the stimulus intensity and the type of the low-frequency pulse current in response to the switching operation received, and causes the pad unit to supply the notification pulse current that changes in response to at least one of the stimulus intensity and the type of the low-frequency pulse current after switching.

9. The low-frequency treatment device according to claim 3, wherein
the operation unit is configured to receive a switching operation configured to switch at least one of a stimulus intensity and a type of the low-frequency pulse current to be supplied, and
the control unit switches at least one of the stimulus intensity and the type of the low-frequency pulse current in response to the switching operation received, and causes the pad unit to supply the notification pulse current that changes in response to at least one of the stimulus intensity and the type of the low-frequency pulse current after switching.

10. The low-frequency treatment device according to claim 4, wherein
the operation unit is configured to receive a switching operation configured to switch at least one of a stimulus intensity and a type of the low-frequency pulse current to be supplied, and
the control unit switches at least one of the stimulus intensity and the type of the low-frequency pulse current in response to the switching operation received, and causes the pad unit to supply the notification pulse current that changes in response to at least one of the stimulus intensity and the type of the low-frequency pulse current after switching.
